(19) United States
(12) Patent Application Publication
(10) Pub. No.: US 2005/0010077 A1
(43) Pub. Date: Jan. 13, 2005

(54) Low flow atrial-arterial shunt for pump-assisted myocardial revascularization without cardiopulmonary bypass

(76) Inventor: Moises Calderon, Huixguilucan (MX)

Correspondence Address: HOLLAND & BONZAGNI, P.C., 171 DWIGHT ROAD, SUITE 302, LONGMEADOW, MA, 01106-1700, US

(21) Appl. No.: 10/768,728

(22) Filed: Jan. 29, 2004

Related U.S. Application Data
(60) Provisional application No. 60/443,411, filed on Jan. 29, 2003.

(51) Int. Cl. A61M 1/12

(52) U.S. Cl. 600/16; 604/9

(57) ABSTRACT

A low flow atrial-arterial shunt for pump-assisted myocardial revascularization without cardiopulmonary bypass comprises a short section of cardiopulmonary bypass tubing securely terminated at either end with a vented cannula adapter. The shunt is used in conjunction with venous and aortic cannulae and one of the conventional peristaltic pumps found in a medical facility's existing cardiopulmonary bypass machine. In use, e.g., for a left ventricular assist, the venous cannula is surgically attached to the left atrium, and the aortic cannula is attached to the aorta. Subsequently, the shunt is attached to the two cannulae and is primed to remove air. Next, the tubing is placed in the peristaltic pump, and the pump is activated, causing blood to flow from the left atrium, through the shunt—where it is propelled along by the pump—and into the aorta.

### This application claims priority from a Provisional Application, Ser. No. 60/443,411, filed Jan. 29, 2003.

### FIELD OF THE INVENTION

The present invention relates to surgical devices and methods, and, more particularly, to devices and methods for temporarily assisting the heart in pumping blood.

### BACKGROUND

In 1953, Dr. John Gibbon of Philadelphia performed the first successful intracardiac operation with the aid of a heart-lung machine, opening a new era for cardiac surgery and establishing the basis for extracorporeal circulation and circulatory support. The heart-lung machine (also known as the cardiopulmonary bypass machine) is an apparatus that functions in place of a patient's heart and lungs during open-heart operations, providing adequate blood flow and perfusion to the rest of the body, while the heart and lungs are arrested. Its basic components are a pump, which provides flow to the blood, and an oxygenator, which oxygenates and removes CO₂ from venous blood. The machine may also include a blood reservoir and a blood temperature regulator.

The most commonly used blood pump in cardio-pulmonary bypass machines is the peristaltic (“roller”) pump. The peristaltic pump consists of two rollers, 180° apart, attached to a central, rotating hub, that are rotated along an arc-shaped (approximately 200°) metal raceway. Plastic tubing between ¼″ and ⅝″ is placed between the rollers and raceway so that the rollers barely occlude the tubing at 180 mmHg back-pressure. As the rollers are rotated along the raceway, one of the rollers successively compresses the tubing, while the opposite roller releases the tubing, so that blood within the tubing is continually propelled in one direction. Put another way, as the rollers are rotated or rolled along the tubing in one direction, the tubing is slightly compressed in a wave-like motion, with blood in the tubing flowing along with the wave-like compression.

While heart-lung machines and associated surgical techniques were a significant medical advancement in their day, medical professionals have come to realize that significant drawbacks. For example, the pump, oxygenator, and other equipment can damage blood cells, while the large size of the machine and its distance from the patient mean a lot of extra blood. Additionally, the chances for an embolism are increased, and it is typically necessary to administer Heparin (an anti-coagulant) to the patient, which can lead to blood loss. Hypothermia may also result.

Accordingly, medical professionals have developed a less invasive procedure for open-heart surgery where the heart is not stopped. Instead, surgery is performed while the heart is still beating, thereby eliminating the need for a heart-lung machine and its associated drawbacks. Since the heart is still beating during surgery, a stabilizer is used to keep movement of the heart to a minimum. The stabilizer is typically some sort of arm attached to a suture plate, which rests on or near the area of operation, for purposes of minimizing movement at that area. However, the stabilizer also limits the action of the heart as a pump, i.e., the heart cannot pump as much or as well because it is partially compressed by the stabilizer. Additionally, the blood vessels proximate the heart may be twisted if the heart has to be temporarily repositioned, further exacerbating the reduction in blood flow.

To overcome this problem, ventricular assist devices are used to help supplement the heart's pumping action during such surgery. These devices typically comprise a pair of long tubes and a centrifugal pump, which has a rapidly rotating impeller (either concentric cones or blades) within a blood compartment. The impeller causes blood to rotate at high speed within the compartment, with the centrifugal force propelling the blood forward through the pump outlet. In use, the tubes are attached to either the left side of the heart (a left ventricular assist device) or to the right side of the heart (a right ventricular assist device) “in parallel,” i.e., the pump supplements the heart's pumping action but does not completely bypass it, and the pump is activated.

Other ventricular assist devices have also been developed. These include axial flow pumps for temporary insertion directly into the heart. Such pumps are based on the Archymides' Principle, where a rod with helical blades is rotated inside a tube to displace liquid. In use, a catheter-mounted, miniature axial flow pump is appropriately positioned inside the heart, and is caused to operate via some sort of external magnetic drive or other appropriate mechanism. With high enough RPM's, a significant amount of blood can be pumped.

While axial flow pumps (and centrifugal pumps to a lesser degree) offer the best overall performance for ventricular assist devices, they suffer from two major drawbacks: scarcity and price. More specifically, axial flow pump- and centrifugal pump-based ventricular assist devices are largely still in the development stage, and/or have not received FDA approval. Accordingly, they are not yet widely available for current use. Moreover, such devices typically require sophisticated drive units and monitoring circuits, which render them quite expensive. Because of these two factors, many hospitals will not be able to purchase and/or use any of the more advanced ventricular assist devices. Instead, they will continue to rely on existing (and almost universally available) technology for major cardiac surgery, where the heart is bypassed with a heart-lung machine and arrested.

Accordingly, a primary object of the present invention is to provide a ventricular assistance device that is partially implemented using existing, widespread medical machinery, that is very inexpensive, and that is easy and effective to use in practice.

### SUMMARY

A low flow atrial-arterial shunt for pump-assisted myocardial revascularization without cardiopulmonary bypass comprises a short section of cardiopulmonary bypass tubing securely terminated at either end with a vented cannula adapter. The shunt is used in conjunction with a venous cannula, an aortic cannula, and a conventional peristaltic or roller pump, which is preferably one of the peristaltic pumps found in a medical facility's existing cardiopulmonary bypass machine. The shunt, cannulae, and pump can be used for left ventricular assist or right ventricular assist, or two shunts can be used for biventricular assist.

In use, e.g., for a left ventricular assist, the venous cannula is surgically attached to the left atrium, and the aortic cannula is attached to the aorta. Subsequently, the shunt is attached to the two cannulae and is primed to remove air. Next, the tubing is placed in the peristaltic pump (which was previously moved close to the patient), and the pump is activated. Blood flows from the left atrium, through the shunt, where it is propelled along by the pump, and into the aorta. In this manner, the pump acts in parallel to the heart's pumping action, which allows for myocardial revascularization without cardiopulmonary bypass. Also, because a medical facility's existing peristaltic pump is used in conjunction with the shunt, which is both inexpensive to manufacture and fully disposable, medical professionals are able to perform ventricular-assisted surgery without having to invest in expensive ventricular assist devices.

### BRIEF DESCRIPTION OF THE DRAWINGS

These and other features, aspects, and advantages of the present invention will become better understood with respect to the following description, appended claims, and accompanying drawings, in which:

FIG. 1 is a side elevation view of a low flow atrial-arterial shunt for pump-assisted myocardial revascularization without cardiopulmonary bypass, according to the present invention;

FIG. 2A is a side elevation view of a vented cannula adapter portion of the atrial-arterial shunt, in longitudinal cross-section, to approximate scale;

FIG. 2B is an end view of the cannula adapter, from the perspective of line 2B-2B in FIG. 2A;

FIG. 3 is a cross-sectional elevation view of a vent cap portion of the cannula adapter shown in FIG. 2A;

FIG. 4 is a conceptual or schematic view of the human heart showing major anatomical features and the path and direction of blood flow;

FIG. 5 is a schematic view of the atrial-arterial shunt in use for a left ventricular assist according to the present invention, with the heart being shown diagrammatically for simplicity; and

FIG. 6 is a schematic view of the atrial-arterial shunt in use for a right ventricular assist according to the present invention.

### DETAILED DESCRIPTION

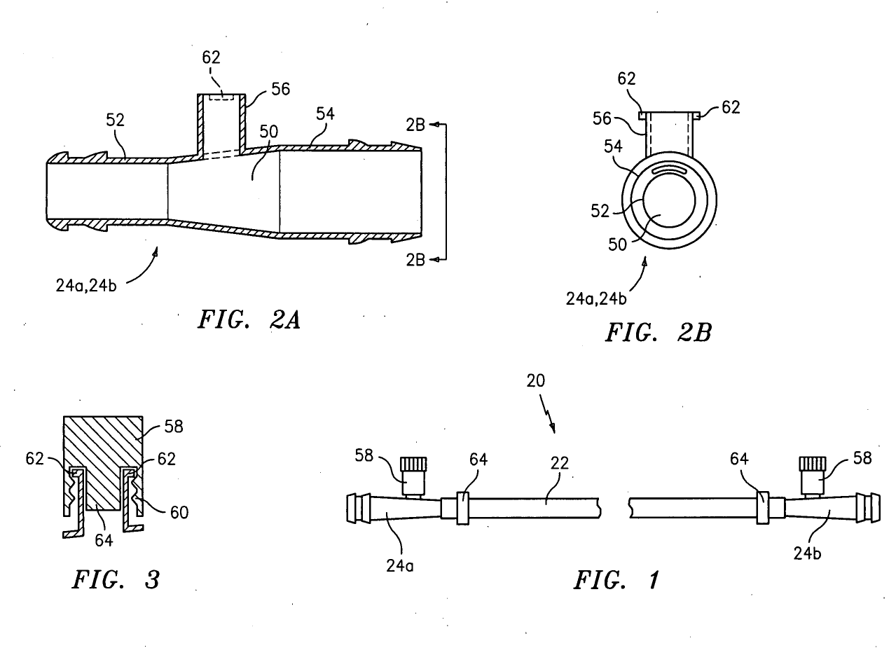

With reference to FIGS. 1-6, a low flow atrial-arterial shunt 20 for pump-assisted myocardial revascularization without cardiopulmonary bypass, according to the present invention, comprises a short section of ¼″×1/16″ (6.4 mm×1.6 mm) conventional cardiopulmonary bypass tubing 22 securely terminated at either end with a vented cannula adapter 24a, 24b. The shunt 20 is used in conjunction with a conventional venous cannula 26, a conventional aortic cannula 28, and a conventional peristaltic or roller pump 30, which is preferably one of the peristaltic pumps found in a medical facility's existing cardiopulmonary bypass machine. The shunt 20, two cannulae 26, 28, and pump 30 can be used for left ventricular assist or right ventricular assist, or two shunts 20 can be used for biventricular assist (along with four cannulae and one or two pumps).

In use, e.g., for a left ventricular assist, the venous cannula 26 is conventionally surgically attached to the left atrium 40 of a patient's heart 42, i.e., in a location and position suitable for adequate blood flow. Similarly, the aortic cannula 28 is conventionally surgically attached to the patient's aorta 44. Subsequently, the shunt 20 is attached to the two cannulae 26, 28, and the shunt 20 and cannulae 26, 28 are primed to remove air. Next, a middle portion of the tubing 22 is placed in the peristaltic pump 30, and the pump 30 is activated. Blood flows from the left atrium 40, through the shunt 20, where it is propelled along by the pump 30, and into the aorta 44. In this manner, the pump 30 acts in parallel to the heart's pumping action, which allows for myocardial revascularization without cardiopulmonary bypass.

As should be appreciated, because a medical facility's existing peristaltic pump 30 is used in conjunction with the shunt 20, which is both inexpensive to manufacture and fully disposable, medical professionals are able to perform ventricular-assisted, non-cardiopulmonary bypass cardiac surgery without having to invest in expensive or exotic ventricular assist devices.

FIGS. 1-3 show the shunt 20 in some detail. As mentioned above, the shunt 20 is a section of tubing 22 terminated at either end by the vented cannula adapters 24a, 24b. The section of tubing 22 is a short length (approximately 6 ft/2 m) of ¼″ inner diameter (“ID”)×1/16″ wall thickness (6.4 mm×1.6 mm) conventional cardiopulmonary bypass tubing, i.e., clear, flexible tubing suitable for sterile medical use. The cannula adapters 24a, 24b are made of translucent, hard plastic, and are cylindrical in overall shape. Each has a longitudinal passageway 50, and includes a ¼″ ID section 52 for attachment to the tubing 22, and a ⅜″ ID section 54 for attachment to the cannulae 26, 28. Both sections 52, 54 are of the luer-lock type for ease of connection, although other connection types may also be used. The cannula adapters 24a, 24b also each include a vent 56, which provides a side opening down through to the passageway 50. The vents 56 are sealed with removable, plastic, knurled caps 58, which include inner threads 60 for securely engaging a pair of protuberances 62 on the vents 56, and a neck plug 64 for enhanced sealing.

To assemble the shunt 20, the tubing 22 is cut to the appropriate length, and the adapters 24a, 24b are connected to the tubing 22 by inserting the ¼″ ID sections 52 into the ends of the tubing. The tubing 22 is then tightly secured to the adapters 24a, 24b by way of plastic cable clamps 64 positioned between the luer-lock flanges. Lastly, the shunt 20 is sterile packaged, e.g., in a standard, sealed, disposable, easily-opened container such as a pouch, either by itself or with another shunt for biventricular assist. (Because such sterile packaging is well known, it is not shown in the drawings).

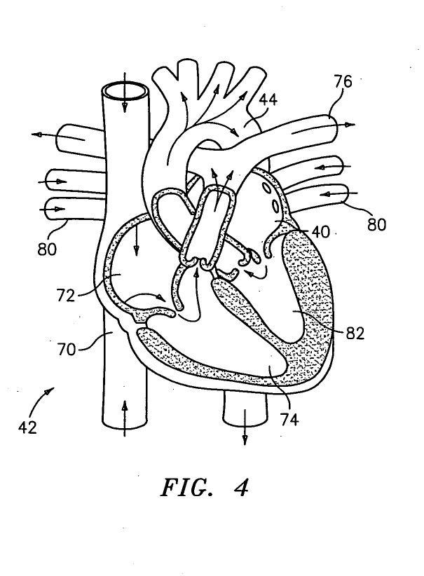

As indicated above, the shunt 20 is not configured for direct connection to the heart 42. Instead, it interconnects the two cannulae 26, 28, which are themselves connected to the heart 42 in a standard manner. To understand proper positioning of the cannulae 26, 28, reference is made to FIG. 4, which is a simplified view of the human heart. Flow arrows indicate direction of blood flow.

Put simply, the human heart 42 is a muscular organ that acts as a pump for moving blood through the human body. Blood is delivered to the heart by veins, and away from the heart by arteries. More specifically, O₂-deficient, CO₂-rich blood is channeled to the heart from the upper and lower body by the superior and inferior vena cavas 70. The blood enters the right atrium 72, passes through to the right ventricle 74, and then up through the pulmonary artery 76 to the lungs 78 for respiration. From the lungs 78, blood travels through the pulmonary veins 80 to the left atrium 40, into the left ventricle 82, and into the aorta 44 for distribution to the body. Of course, the chambers (atriums and ventricles) act in coordinated effort to effectuate a proper pumping action.

In traditional cardiac surgery, the heart is completely bypassed. In ventricular assisted surgery, the heart's action is augmented by placing a pump “in parallel” to the right side of the heart (right ventricular assist), the left side (left ventricular assist), or both (biventricular assist). For the right side of the heart 42, blood enters the right atrium 72 and exits through the pulmonary artery 76, with the right atrium 72 and right ventricle 74 providing motive force. Accordingly, for a right ventricular assist, the pump is connected between the right atrium 72 (entrance) and pulmonary artery 76 (exit). Similarly, for a left ventricular assist, the pump is connected between the left atrium 40 (entrance) and aorta 44 (exit). Of course, in both cases the pump is set up to pump blood in the same direction as the normal blood flow through the heart.

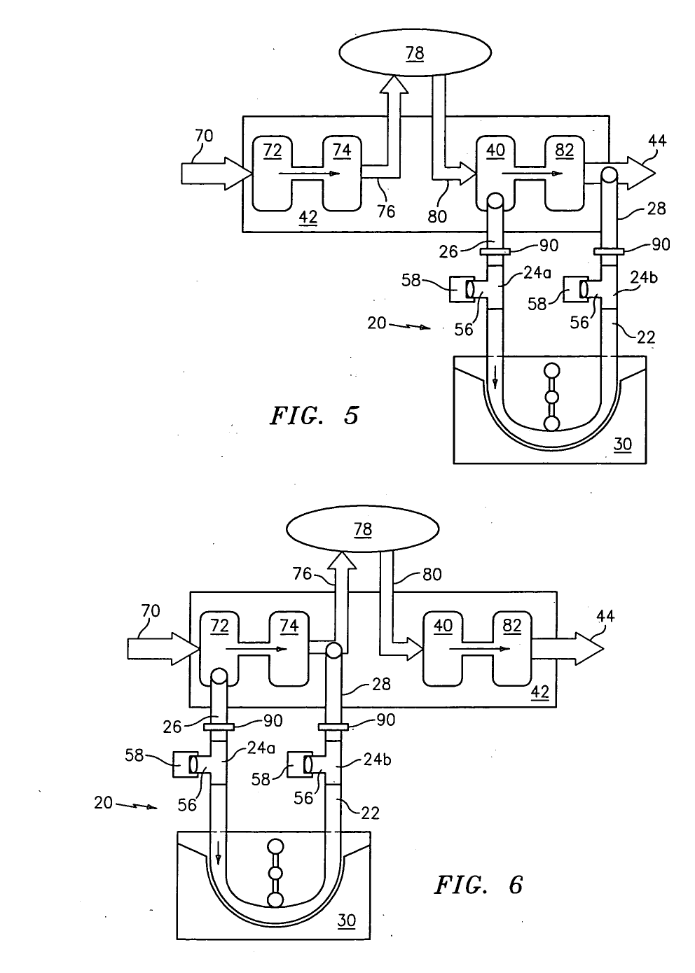

FIGS. 5 and 6 show how the shunt 20 is positioned, according to the present invention, in more detail. In FIG. 5, the shunt 20 is being used for a left ventricular assist (note that the heart 42 is shown diagrammatically). As such, a conventional venous cannula 26 is connected to the left atrium 40, and a conventional aortic cannula 28 is connected to the aorta 44. The shunt 20 interconnects the two cannulae 26, 28, and is routed through a peristaltic pump 30. In use, the heart 42 continues to pump, with blood flowing in the direction indicated. However, some of the blood entering the left atrium 40 passes into the venous cannula 26, through the shunt 20, through the aortic cannula 28, and into the aorta 44, where it continues on to the rest of the patient's body. As the blood passes through the shunt 20, it is moved along gently by the pump 30, which thereby augments the pumping action of the heart. More specifically, with the shunt 20 and pump 30 in place, the patient's body receives an adequate supply of blood even if the heart 42 is not pumping as much as usual because of the ongoing medical procedure, and without the need for a cardiopulmonary bypass machine.

To use the shunt 20, the patient is prepped and his or her heart is accessed, according to standard medical practices. This may include the IV administration of Heparin at a dose of 10,000 IU.

Next, the venous cannula 26 (a 20Fr, angled, conventional venous cannula) is conventionally surgically attached to the left atrium 40 (assuming a left ventricular assist), and the aortic cannula 28 (a 22Fr conventional aortic cannula) is conventionally surgically attached to the aorta 44. Both cannulae 26, 28 are purged during the surgical connection process to prevent air from entering the blood stream, if necessary, and both are initially clamped with standard clamps 90.

Next, the vented cannula adapters 24a, 24b are securely attached to the cannulae 26, 28, respectively.

Subsequently, the shunt 20 is primed. To do so, the clamp 90 on the venous cannula 26 is left in place, the cap 58 on the adapter 24a is opened or removed, and the clamp 90 on the aortic cannula 28 is unclamped. Since the aorta end is at a higher pressure than the left atrium end, blood flows from the aorta 44 through the tubing 22 and out the vent 56 on the adapter 24a. This forces the air in the tubing 22 out through the vent 56. The tubing 22 may be physically manipulated to dislodge any trapped air bubbles. Subsequently, the cap 58 is put back in place, and the clamp 90 on the aortic cannula 28 may be reclamped.

Next, the shunt is placed in the pump 30. As mentioned above, most cardiopulmonary bypass machines, such as those found in most medical facilities, have one or more standard peristaltic pumps 30. These pumps are typically modular, such that they can be removed from the bypass machine and used remotely. According to the present invention, one of pumps 30 is removed from a bypass machine, and is set up near the patient (before the operation, of course). In fact, the shunt 20 is purposefully made short so that the distance between the patient and the pump 30 is necessarily minimized. This reduces the volume of blood needed to use the pump, thereby eliminating the need for extra blood, minimizing the pump's effect on the blood, and increasing the pump's effectiveness.

With the pump 30 in place near the patient, the middle part of the shunt tubing 22 is placed in the pump 30, i.e., with the tubing lying against the pump raceway and the pump roller head against the tubing. The pump 30 is closed or otherwise prepared for use. Then, the clamps 90 on the cannulae 26, 28 are unclamped, and the pump 30 is activated. At some point during the process, it may be necessary to use the vent on the adapter 24b to further purge the tubing 22 of air.

For most surgical procedures, the peristaltic pump 30 should be set to pump at a rate from about 2 liters/minute to about 3 liters/minute.

Once the pump 30 is up and running, the main surgical procedure is performed. Once completed, the pump 30 is stopped, the cannulae are removed in a conventional manner, and the Heparin is reversed. The operation is then completed as normal.

FIG. 6 shows how the shunt 20 and associated equipment are positioned for a right ventricular assist. Essentially, the procedure is the same as described above with respect to FIG. 5, except that the venous cannula 26 is conventionally surgically attached to the right atrium 72 and the aortic cannula 28 is conventionally surgically attached to the pulmonary artery 76.

For biventricular assist, two shunts 20 are used, one in the manner as shown in FIG. 5, and one in the manner as shown in FIG. 6. Two peristaltic pumps 30 can be used, one for each shunt 20, or a single peristaltic pump 30 can be used, provided it is capable of holding and acting on two tubes 22, e.g., pumping around 3 liters/minute on both lines.

Using the above-described procedures on four patients, all exhibited early extubation, reduced stay in the ICU, and early discharge from the hospital. No operative complications or mortality were observed, and at three months follow-up, the patients continued to rehabilitate uneventfully. These results demonstrate that the present invention is safe and efficient, and especially cost-effective in comparison to axial- and centrifugal-flow based devices developed to support “off the pump” aortocoronary bypass operations.

To summarize, the method or procedure according to the present invention can be characterized as a method of pump-assisting a patient's heart for myocardial revascularization without cardiopulmonary bypass, where, for a left ventricular assist, the method or procedure comprises the steps of: (i) surgically attaching a first cannula to the aorta; (ii) surgically attaching a second cannula to the left atrium; (iii) interconnecting the first and second cannulae with an atrial-arterial shunt, wherein the shunt comprises a length of clear, flexible tubing terminated at one end by a first adapter and at the other end by a second adapter, with each adapter having a selectively closable vent, and with the first and second adapters being respectively connected to the first and second cannulae; (iv) priming the shunt to remove air; (v) operatively inserting the shunt tubing into a peristaltic pump, wherein: the peristaltic pump is one of the peristaltic pumps found in a medical facility's existing cardiopulmonary bypass machine; and the pump is placed near the patient, i.e., within a few feet (1 m) of the patient; (vi) allowing unrestricted blood flow through the two cannulae and shunt; and (vii) activating the peristaltic pump to pump blood through the shunt and in parallel to the heart's pumping action.

As should be appreciated, the step of priming the pump to remove air can include the steps of: (i) opening one or both vents and allowing blood to flow into the shunt via one or both of the cannulae, where the blood displaces air in the shunt and forces it out one or both vents; and (ii) closing the open vents (or closing both vents if both were opened) once the air in the tubing has been removed.

Also, for a right ventricular assist, the method according to the present invention would include the substitute steps of surgically attaching the first cannula to the pulmonary artery and surgically attaching the second cannula to the right atrium. Also, in the case of a biventricular assist, the method would include the steps of: (i) respectively attaching four cannulae to the pulmonary artery, aorta, right atrium, and left atrium; (ii) interconnecting the left atrium and aorta with a first shunt, and interconnecting the right atrium and pulmonary artery with a second shunt; (iii) priming both shunts; (iv) operatively inserting the shunts' tubing into one or two of the peristaltic pumps found in a medical facility's existing cardio-pulmonary bypass machine; and (v) after allowing for unrestricted blood flow through the cannulae and shunts, starting the pumps.

Since certain changes may be made in the above-described low flow atrial-arterial shunt for pump-assisted myocardial revascularization without cardiopulmonary bypass, without departing from the spirit and scope of the invention herein involved, it is intended that all of the subject matter of the above description or shown in the accompanying drawings shall be interpreted merely as examples illustrating the inventive concept herein and shall not be construed as limiting the invention.

Having thus described the invention,

1. An atrial-arterial shunt for pump-assisted myocardial revascularization without cardiopulmonary bypass comprising:
a. a section of translucent tubing having first and second ends and an interior; and
b. first and second cannula adapters attached to the first and second ends of the tubing, respectively, each of said cannula adapters comprising:
i. a body having a passageway and first and second ends, wherein the first end of the cannula adapter body is attached to the section of tubing such that the cannula adapter passageway is in fluid communication with the interior of the tubing, and wherein the second end of said cannula adapter body is adapted for attachment to a cannula; and
ii. a vent extending through the cannula adapter body from the passageway through to an exterior of the cannula adapter body, wherein the vent is sealable for selectively opening and closing the vent for priming purposes.

2. The shunt of claim 1 further comprising a cap removably attached to the vent of each cannula adapter for selectively opening and closing the vent for priming purposes.

3. The shunt of claim 1 wherein the section of tubing has a length of no more than two meters, to reduce the amount of blood required to fill the shunt for use in pump-assisted myocardial revascularization.

4. A system for pump-assisted myocardial revascularization without cardiopulmonary bypass comprising:
a. a sealed, openable container having a sterile interior; and
b. a first sterile atrial-arterial shunt sealed inside the container and comprising:
i. a section of translucent tubing having first and second ends and an interior; and
ii. first and second cannula adapters attached to the first and second ends of the tubing, respectively, each of said cannula adapters comprising:
a body having a passageway and first and second ends, wherein the first end of the cannula adapter body is attached to the section of tubing such that the cannula adapter passageway is in fluid communication with the interior of the tubing, and wherein the second end of the cannula adapter body is adapted for attachment to a cannula; and
a vent extending through the cannula adapter body from the passageway through to an exterior of the cannula adapter body, wherein the vent is sealable for selectively opening and closing the vent for priming purposes.

5. The system of claim 4 wherein the atrial-arterial shunt further comprises two caps respectively removably attached to the vents of the cannula adapters for selectively opening and closing the vents for priming purposes.

6. The system of claim 4 further comprising a peristaltic pump configured to accommodate the shunt tubing for pumping purposes, wherein the peristaltic pump is one of a medical facility's existing peristaltic pumps from a cardiopulmonary bypass machine.

7. The system of claim 6 wherein the shunt's section of tubing has length of no more than two meters, to reduce the amount of blood required to fill the shunt for use in pump-assisted myocardial revascularization.

8. The system of claim 4 further comprising a second sterile atrial-arterial shunt sealed in the container and generally identical to the first shunt.

9. The system of claim 8 wherein the atrial-arterial shunts further comprise a plurality of caps respectively removably attached to the cannula adapter vents for selectively opening and closing the vents for priming purposes.

10. The system of claim 8 wherein the sections of tubing of each shunt have a length of no more than two meters, to reduce the amount of blood required to fill the shunts for use in pump-assisted myocardial revascularization.

11. The system of claim 8 further comprising a peristaltic pump configured to accommodate the shunt tubing for pumping purposes, wherein the peristaltic pump is one of a medical facility's existing peristaltic pumps from a cardiopulmonary bypass machine.

12. The system of claim 11 wherein the sections of tubing of each shunt have a length of no more than two meters, to reduce the amount of blood required to fill the shunts for use in pump-assisted myocardial revascularization.

13. A method of pump-assisted myocardial revascularization without cardiopulmonary bypass comprising the steps of:
a. surgically attaching a first cannula to the aorta of a patient's heart;
b. surgically attaching a second cannula to the left atrium of the patient's heart;
c. interconnecting the first and second cannulae with a first sterile atrial-arterial shunt, wherein:
i. the shunt comprises a section of translucent tubing terminated at a first end by a first cannula adapter and at a second end by a second cannula adapter;
ii. each cannula adapter has a vent, said vent being sealable for selectively opening and closing the vent for priming purposes; and
iii. the first and second cannula adapters are respectively connected to the first and second cannulae;
d. priming the first shunt to remove air;
e. inserting the first shunt tubing into a first peristaltic pump, wherein the first peristaltic pump is one of a medical facility's existing peristaltic pumps from a cardiopulmonary bypass machine; and
f. activating the first peristaltic pump to pump blood through the shunt and in parallel to the patient's heart's pumping action.

14. The method of claim 13 wherein the step of priming the shunt to remove air comprises the sub-steps of: opening the vent on the first cannula adapter; allowing blood to flow through the second cannula from the left atrium through the tubing to the first cannula adapter, wherein the flowing blood forces the air out of the tubing and through the vent on the first cannula adapter; and closing the vent on the first cannula adapter.

15. The method of claim 13 wherein: the section of translucent tubing is no longer than two meters, to reduce the amount of blood required to fill the shunt for use in pump-assisted myocardial revascularization; and the method further comprises the step, prior to step e), of moving the first peristaltic pump within one meter of the patient.

16. The method of claim 13 further comprising the steps of:
a. surgically attaching a third cannula to the pulmonary artery of the patient's heart;
b. surgically attaching a fourth cannula to the right atrium of the patient's heart;
c. interconnecting the third and fourth cannulae with a second sterile atrial-arterial shunt generally identical to the first shunt;
d. priming the second shunt to remove air;
e. inserting the second shunt's tubing into a selected one of the first peristaltic pump or a second peristaltic pump that is also one of a medical facility's existing peristaltic pumps from a cardiopulmonary bypass machine; and
f. activating the selected one of the first peristaltic pump or the second peristaltic pump to pump blood through the second shunt and in parallel to the pumping action of the patient's heart.

17. The method of claim 16 wherein: the section of translucent tubing of each shunt is no longer than two meters, to reduce the amount of blood required to fill the shunts for use in pump-assisted myocardial revascularization; and the method further comprises the step of moving the peristaltic pump(s) within one meter of the patient.

18. A method of pump-assisted myocardial revascularization without cardiopulmonary bypass comprising the steps of:
a. surgically attaching a first cannula to a first location on a patient's heart;
b. surgically attaching a second cannula to a second location on the patient's heart;
c. interconnecting the first and second cannulae with a sterile atrial-arterial shunt, wherein:
i. the shunt comprises a section of translucent tubing terminated at a first end by a first cannula adapter and at a second end by a second cannula adapter;
ii. each cannula adapter has a vent, said vent being sealable for selectively opening and closing the vent for priming purposes; and
iii. the first and second cannula adapters are respectively connected to the first and second cannulae;
d. priming the shunt to remove air;
e. inserting the shunt tubing into a peristaltic pump, wherein the peristaltic pump is one of a medical facility's existing peristaltic pumps from a cardiopulmonary bypass machine; and
f. activating the peristaltic pump to pump blood through the shunt; wherein:
g. the first and second locations on the patient's heart are chosen so that when the peristaltic pump is activated it acts to operably pump blood in parallel to the pumping action of the patient's heart.

19. The method of claim 18 wherein: the section of translucent tubing has a length of no more than two meters, to reduce the amount of blood required to fill the shunt for use in pump-assisted myocardial revascularization; and the method further comprises the step, prior to step e), of moving the peristaltic pump within one meter of the patient.
